(19) United States
(12) Patent Application Publication
(10) Pub. No.: US 2025/0320560 A1
(43) Pub. Date: Oct. 16, 2025

(54) METHOD OF TREATING OBESITY WITH PRECISION MEDICINE PANEL

(71) Applicants: Justin Jacob FARAJZADEH, San Diego, CA (US); Yadira A. VALLES, Woodland Hills, CA (US)

(72) Inventors: Justin Jacob FARAJZADEH, San Diego, CA (US); Yadira A. VALLES, Woodland Hills, CA (US)

(21) Appl. No.: 19/176,898

(22) Filed: Apr. 11, 2025

Related U.S. Application Data
(60) Provisional application No. 63/633,190, filed on Apr. 12, 2024.

(51) Int. Cl. C12Q 1/6883; G16H 10/40

(52) U.S. Cl. CPC C12Q 1/6883; G16H 10/40; C12Q 2600/106; C12Q 2600/156; G01N 2800/044; G01N 2800/52

(57) ABSTRACT

The present invention provides a method for precision anti-obesity therapy, utilizing a proprietary panel of genetic variants to predict individual patient response to weight-loss medications as well as a related companion diagnostic. In particular, the method analyzes variants in genes including GLP-1 R, CNR1, TCF7L2, DPP4 and others, which have established associations with drug efficacy and metabolism in obesity treatment. By genotyping these markers, the method guides selection and dose optimization of specific anti-obesity medications—such as GLP-1 receptor agonists, metformin, SGLT2 inhibitors, and DPP4 inhibitors—tailored to the patient's genetic profile. Notably, GLP-1R polymorphisms (e.g., rs6923761) are leveraged as especially predictive indicators of enhanced weight loss response to GLP-1 receptor agonists. Through this innovative genetic profiling approach, the invention enables personalized treatment strategies that maximize efficacy, minimize trial-and-error in drug choice, and reduce adverse effects, thereby embodying the principles of precision medicine in obesity care.

### CROSS-REFERENCE TO RELATED APPLICATIONS

The present application claims priority to provisional patent application 63/633,190, filed on Apr. 12, 2024, the entire contents of which are incorporated herein by reference.

### BACKGROUND OF THE INVENTION

Obesity and related metabolic disorders present a global health challenge, with patients often exhibiting variable responses to standard weight-loss therapies. A range of pharmacological treatments is available for obesity—including GLP-1 receptor agonists, biguanides like metformin, SGLT2 inhibitors, DPP4 inhibitors, and combination therapies (e.g., naltrexone/bupropion and phentermine/topiramate)—yet their efficacy and tolerability can differ greatly among individuals. This variability is frequently observed in clinical practice, where one patient may experience substantial weight loss on a given medication while another has minimal response or intolerable side effects. The present disclosure is directed to overcoming these and other deficiencies,

### BRIEF SUMMARY OF THE INVENTION

This panel comprises 16+ gene variants (single nucleotide polymorphisms, SNPs) that collectively inform the likely efficacy and safety of various anti-obesity medications for an individual patient. By testing a patient's DNA for these variants, a clinician can predict responses to medications such as:
i, GLP-1 Receptor Agonists (GLP-1 RAs)—e.g., liraglutide, semaglutide
ii. SGLT2 Inhibitors—e.g., canagliflozin, empagliflozin
iii. DPP-4 Inhibitors—e.g., sitagliptin, linagliptin
iv. Biguanides—metformin
v, Combination therapies—e.g., naltrexone-bupropion (NB), phentermine-topiramate.

The panel includes variants in key genetic markers that influence drug pharmacodynamics or pharmacokinetics relevant to obesity and overweight treatment. Examples (with representative SNPs) include:
i. GLP1 R (rs6923761, rs10305420)—affects GLP-1 RA response.
ii. TCF7L2 (rs7903146)—influences response to incretin therapies and metformin.
iii. CNR1 (rs1049353)—linked to weight loss efficacy of GLP-1 RAs via endocannabinoid pathways.
iv. DPP4 (rs2909451, rs759717)—affects efficacy of DPP-4 inhibitors by altering DPP-4 enzyme activity.
v. SLC47A1 (MATE1, rs2289669)—modulates metformin pharmacokinetics and glycemic response.
vi. ATM (C11 orf65 locus, rs11212617)—associated with metformin treatment success [symbol] 31†L19-L279** (rs72551330, defining UGT1A9*3)—alters metabolism of SGLT2 inhibitors (e.g., canagliflozin).
vii. OCT1 (SLC22A1, e.g., rs628031)—influences metformin absorption and action [symbol] 32†L1-L8.
viii. CYP2B6 (399499 or *star allele defining *6)—affects bupropion metabolism, relevant for NB combination.
ix. GRIK1 (rs2832407)—relates to topiramate response, possibly modulating efficacy in combination therapy.
x. DRD2/ANKK1 (rsl800497 A1 allele)—dopamine D2 receptor availability, A1+ allele predicts greater weight loss on NB therapy.
xi. OPRM1 (rs1799971 A118G)—μ-opioid receptor variant; may influence naltrexone response (e.g., G allele linked to better naltrexone effect in some studies).
xii. MC4R (common obesity alleles)—indicates a genetic predisposition to hyperphagia and severe obesity; can suggest need for aggressive appetite suppression.
xiii. FTO (e.g., rs9939609)—fat mass and obesity-associated gene; risk alleles correlate with higher BMI and perhaps different NB responses when combined with DRD2 genotype.
xiv. GNB3 (rs5443, C825T)—G-protein p3 subunit; T allele linked to better weight loss on sibutramine (historically) [symbol] 34†L3615-L3623 [symbol] and p for stimulant-based or noradrenergic drugs.

### DETAILED DESCRIPTION OF THE INVENTION

Differences in drug response have been increasingly linked to genetic factors, as evidenced by pharmacogenetic studies in metabolic and endocrine disorders. Recent research has identified specific gene variants that influence how patients metabolize medications or respond to their therapeutic effects. For example, a common variant in the TCF7L2 gene (rs7903146), which is a well-known risk factor for type 2 diabetes, has been shown to alter incretin signaling and affect responses to glucose-lowering drugs. In the context of metformin (a first-line anti-diabetic often used for weight management in insulin-resistant patients), polymorphisms in transporter and enzyme genes have demonstrated clinical significance. A notable case is the SLC47A1 gene (encoding the MATE1 transporter): the rs2289669 G>A variant has been associated with improved glycemic response to metformin therapy, as confirmed by meta-analysis (patients carrying the A allele had greater HbA1c reduction). Likewise, a genome-wide study identified a variant near the ATM gene (rs11212617) that correlates with metformin treatment success (odds ratio ˜1.35 for achieving glycemic targets). These findings underscore that underlying genetics can markedly influence the pharmacodynamics and pharmacokinetics of anti-obesity and anti-diabetic medications.

Despite such insights, current clinical practice in obesity treatment still largely relies on trial-and-error prescribing or generalized guidelines, without routine genetic screening. This can lead to suboptimal outcomes—patients might endure ineffective therapy for months or suffer avoidable side effects before a more suitable regimen is found. There is a clear, unmet need for a precision medicine approach to obesity: one that tailors medication choices to the individual's biological makeup. By harnessing pharmacogenetics, it becomes possible to predict drug sensitivity or resistance before treatment begins, enabling clinicians to choose the right medication (and dose) for the right patient from the outset.

Some prior efforts in personalized medicine have focused on single genes or limited markers (for instance, assessing TCF7L2 status to inform diabetes medication choice). However, obesity is a multifactorial condition influenced by numerous pathways—from gut hormone signaling and neuroreceptor activity to metabolism and fat storage. Therefore, a more comprehensive genetic panel is required to capture the key determinants of both drug response and the obesity phenotype. The present invention addresses this need by providing a method for weight-loss pharmacotherapy, wherein the treatment comprises a multi-gene panel specifically designed as a companion diagnostic. In doing so, it bridges the gap between genetic research and clinical application, offering a tool to improve outcomes in the management of obesity and overweight conditions.

The invention comprises a method of treating obesity and overweight comprising a novel multi-gene panel and companion diagnostic kit to personalize anti-obesity pharmacotherapy. By integrating 16 key genetic markers with evidence-based drug response associations, this invention empowers clinicians to make informed decisions, matching patients to optimal treatments such as GLP-1 R agonists, SGLT2 inhibitors, DPP-4 inhibitors, metformin, and combination therapies (naltrexone-bupropion, phentermine-topiramate) in a way that maximizes efficacy, minimizes side effects, and accounts for individual differences in obesity pathophysiology (from metabolic to hedonic drivers). This approach aligns with the cutting edge of precision medicine and addresses a critical need in the management of obesity and overweight-improving outcomes in a condition that has proven challenging with traditional empirical treatment selection.

The invention integrates different markers into a single predictive test. For example, GLP-1 receptor agonists (such as liraglutide and semaglutide) are highly effective for many patients, and variants in the GLP1 R gene have been linked to variability in weight loss and glycemic outcomes. Patients carrying certain GLP1 R alleles (notably the minor A allele of rs6923761) have shown significantly greater delay in gastric emptying and trends toward enhanced weight reduction when treated with GLP-1 agonists, as compared to those without the variant. Similarly, genetic differences in the DPP4 gene (which encodes dipeptidyl peptidase-4, the target of DPP4 inhibitors like sitagliptin) can influence DPP-4 enzymatic activity and treatment efficacy. Variants such as rs2909451 in DPP4 have been associated with different levels of DPP-4 activity under DPP4 inhibitor therapy, potentially affecting how well these drugs improve glycemic control. Beyond glucose-centric therapy, genes affecting reward pathways and drug metabolism are also relevant to obesity pharmacotherapy. For instance, the combination of naltrexone-bupropion (approved for weight loss) acts on the brain's reward and appetite centers; here, polymorphisms in the OPRM1 gene (μ-opioid receptor) and the DRD2/ANKK1 gene (dopamine D2 receptor pathway) may modulate treatment outcomes. The OPRM1 A118G variant is known to alter β-endorphin binding and has been widely studied in addiction medicine for its impact on naltrexone response, suggesting that it could likewise influence how effectively naltrexone mitigates food cravings in obesity. The DRD2 Taq1A polymorphism (rs1800497 in the ANKK1 gene, affecting DRD2 receptor availability) has been linked to hedonic eating and food addiction phenotypes; patients with certain DRD2 risk alleles may have a blunted dopamine reward response and thus differentially benefit from therapies targeting cravings (such as bupropion, which enhances dopamine, or naltrexone, which modulates opioid-rmediated reward).

Another example is the GRIK gene, encoding a kainate glutamate receptor: an intronic variant rs2832407 in GRIK1 has been shown to predict the efficacy of topiramate (an anticonvulsant used off-label for impulse control and included in a combination weight-loss drug) in reducing alcohol consumption, with one genotype group experiencing significantly greater benefit. This suggests GRIK polymorphisms could also influence topiramate's appetite-suppressing effects in obesity treatment. Additionally, metabolism of bupropion (a component of the ContraveC) weight-loss medication) is primarily mediated by the CYP236 enzyme; individuals with the common loss-of-function CYP2B66 allele have higher plasma levels of bupropion's active metabolites and, notably, were shown to have better smoking cessation outcomes on bupropion compared to those without the variant. This implies that CYP2B6 genotypes could affect bupropion's efficacy for weight management as well or at least influence the optimal dosing to achieve desired plasma exposure.

In summary, multiple genetic markers across diverse biological pathways—from hormone receptors and enzymes to neurotransmitter regulators—contribute to how a patient responds to anti-obesity medications. By combining these markers into a single panel, the present invention offers an unprecedented level of personalization in obesity therapy, aiming to significantly improve patient outcomes while reducing the time and cost associated with finding an effective treatment plan.

The invention is directed to a method for treating obesity and overweight comprising predicting patient response to anti-obesity medications and guiding personalized treatment based on a targeted panel of genetic variants. In one aspect, the invention provides a method for treating obesity and overweight comprising a pharmacogenetic test comprising: (a) obtaining a biological sample from a subject (such as a blood or saliva sample); (b) analyzing the sample to determine the subject's genotype for a panel of specified gene variants (particularly in genes GLP-1 R, CNR1, TCF7L2, SLC47A1, ATM (C11orf65), UGTIA9, DPP4, OPRM1, GRIK1, CYP2B6, DRD2, OCT1 (SLC22A1), GNB3, and additional obesity-related loci such as MC4R and FTO); (c) predicting, based on the genotype profile, the efficacy or likely therapeutic response to one or more anti-obesity or weight-loss drugs in that subject; and (d) administering the anti-obesity or weight-loss drugs to the subject. The predicted response can then be used to guide the selection of an optimal medication and its dosage for treating the subject's obesity or overweight condition.

In another aspect, the invention encompasses a companion diagnostic tool or kit incorporating the above method. This tool may include reagents for genotyping the specified variants (for example, a microarray chip or PCR primer panel) and an interpretative guide or software or algorithm that translates the genetic results into clinically actionable recommendations that incorporates data from patients to output a report ranking potential medications. The output of the test is a report that stratifies available treatment options by predicted efficacy and risk for the individual patient. For instance, the report might highlight that a patient has a high likelihood of robust weight loss on a GLP-1 receptor agonist due to a favorable GLP1 R genotype, whereas another medication (e.g., a DPP4 inhibitor) may be less effective given the patient's specific genetic makeup in the incretin pathway.

The genetic panel at the core of this method is a proprietary combination of markers selected for their proven impact on weight-loss drug response. Unlike prior art that may focus on single genes or solely on diabetes control, this invention uniquely integrates multiple genes affecting different drug classes relevant to obesity treatment, as well as genes reflecting the patient's inherent obesity predisposition. By covering pharmacokinetic genes (influencing drug absorption, distribution, metabolism, excretion) and pharmacodynamic genes (influencing drug targets and pathways), the method provides a two-tiered analysis: first, predicting which medication is most likely to be effective and well-tolerated, and second, offering insights into the patient's metabolic and appetite regulation profile which can inform adjunct strategies (such as behavioral interventions or combination therapies).

For example, an innovative aspect of the invention is the emphasis on GLP-1 receptor (GLPIR) gene variants as predictors of treatment success with GLP-1 analogs. GLP-1 analogs (like liraglutide, semaglutide, dulaglutide, etc.) are among the most potent anti-obesity medications currently available and identifying patients who will derive maximal benefit from them has significant clinical value. The inclusion of GLPIR rs6923761 (a missense variant Gly168Ser) in the panel is based on evidence that this variant is an independent predictor of weight reduction and metabolic improvement in response to GLP-1RA therapy.

No existing commercial diagnostic for obesity management specifically uses GLPIR genotype in this manner. Similarly, by incorporating the CNR1 gene (which encodes the cannabinoid receptor 1, involved in appetite regulation) and TCF7L2 (a transcription factor affecting insulin and incretin action), the panel captures additional dimensions of how patients might respond to drugs that modulate appetite or insulin sensitivity.

Among the several advantages of the method of the invention are:
i. Personalized Therapy: Aligns with the precision medicine paradigm, ensuring “the right drug at the right dose for the right patient.”
ii. Improved Efficacy: By matching patients to the drug class most likely to work for them, average weight loss per patient should increase. For example, identifying GLP-1RA responders vs. NB responders vs. metformin responders can direct each to their best option, rather than random trial.
iii. Reduced Side Effects: Patients genetically less likely to respond (and thus who might need high doses) can avoid unnecessary exposure. Conversely, if someone is a likely super-responder, perhaps a lower dose will suffice, reducing side effect risk.
iv. Comprehensive Scope: Unlike single-gene tests, this panel covers multiple pathways: gut hormones, insulin signaling, neural reward, drug metabolism, etc. This is crucial because obesity is multifactorial; the test doesn't hinge on one mechanism but paints a broad picture.
v. Guiding Combination Therapy: The panel can identify when combinations are indicated. For instance, if genetics suggest only moderate response to a GLP-1 RA, but also slight dopamine-related eating, combining a low-dose NB with a GLP-1RA might yield synergy. The report could suggest such personalized combos (with appropriate clinical judgment).
vi. Stratifying Obesity Subtypes: The results can classify patients (e.g., “predominantly metabolic obesity” vs “predominantly hedonic obesity”) based on gene load in certain categories, which aligns with emerging obesity subtype models (like “hungry brain” vs “hungry gut” paradigms).
vii. Utility: Using this genetic panel, healthcare providers can stratify patients by obesity “subtype” and choose the optimal therapy.

The method is broadly applicable to the major categories of anti-obesity pharmacotherapy, including:
i, GLP-1 Receptor Agonists (GLP-1 RAs)—e.g., liraglutide, semaglutide, dulaglutide—guided by variants in GLP1R, CNR1, TCF7L2, etc.
ii. Biguanides—primarily metformin—guided by variants in ATM (C11orf65), SLC47A1 (MATE1), and OCT1 (SLC22A1), which influence metformin's pharmacokinetics and glycemic effect.
iii. SGLT2 Inhibitors—e.g., canagliflozin, empagliflozin—guided by metabolic enzyme variants like UGTIA9 (where the UGTIA93 allele leads to higher drug exposure).
iv. DPP-4 Inhibitors—e.g., sitagliptin, saxagliptin—guided by DPP4 gene polymorphisms and related incretin pathway genes (GLP1 R, TCF7L2).
v. Combination Therapies for Obesity—e.g., naltrexone/bupropion (Contrave@) and phentermine/topiramate (QsymiaC))—guided by neuroreceptor and metabolism genes such as OPRM1, DRD2/ANKK1, CYP2B6, and GRIK1, which influence the efficacy of these combinations on appetite and reward circuitry.

Another aspect of the invention provides methods for stratifying patients into subpopulations based on their genotype profile. For example, patients could be classified as “GLP-1 RA likely responders,” “metformin-preferring phenotype,” “high craving phenotype,” etc., which in turn directs a clinician to prioritize a certain therapeutic strategy (GLP-1 RA, insulin sensitizer, appetite suppressant, etc.).

In some embodiments, the method further comprises recommending a dosing strategy—for instance, if a patient has a genotype indicating slower drug metabolism, a lower starting dose might be advised to avoid side effects, or conversely, a higher dose or more aggressive titration might be recommended if the genotype predicts a blunted response. For example, for a subject carrying a poor-metabolizer genotype for a drug, the method includes starting at a lower dose to avoid toxicity, whereas for a subject with a genotype suggesting rapid metabolism or attenuated drug effect, the method includes starting at a standard or higher initial dose to ensure efficacy.

One embodiment of this invention is a therapeutic treatment comprising a novel testing method that transforms an individual's genetic information into a concrete treatment plan for weight loss. By doing so, it maximizes the therapeutic benefit of anti-obesity medications on a per-patient basis. This not only has the potential to improve patient health outcomes (greater weight loss, better metabolic control) but also to reduce healthcare costs by minimizing ineffective treatments and accelerating the time to find the right therapy.

The invention is described in terms of preferred embodiments, wherein a patient's DNA is analyzed for specific polymorphisms and the resulting information is used to make evidence-based decisions on obesity drug therapy. It should be understood that while the exemplary gene list and drug classes are described with particularity, the method could be expanded to include additional variants or medications as research evolves, without departing from the core inventive concept.

Central to the invention is a proprietary genetic panel comprising a curated list of gene variants that influence obesity treatment. This panel uniquely combines markers related to drug response and markers related to obesity traits to provide a comprehensive genetic assessment. The preferred panel genes and representative variants are summarized below:
i. GLPIR (Glucagon-Like Peptide 1 Receptor)—e.g., variants rs6923761 (Gly16BSer) and rs10305420 in GLP-1 R.
Rationale: GLP-1 R is the target of GLP-1 receptor agonist drugs (like liraglutide and semaglutide). Variations in this receptor can alter drug binding or receptor signaling. Studies have shown that patients carrying the Ser168 (minor A) allele of rs6923761 experience different outcomes on GLP-1 RAs, including a greater delay in gastric emptying and trends toward more weight loss. In one clinical study, the GLP1 R rs6923761 genotype was an independent predictor of changes in body weight and fat mass during 14 weeks of liraglutide therapy. Therefore, GLPIR genotyping can indicate whether a patient is likely to be a “strong responder” to GLP-1 based therapies.
ii. CNR1 (Cannabinoid Receptor 1)—eg., variant rs1049353. Rationale: CNR1 encodes the CB1 receptor, which is involved in regulating appetite and energy balance. Polymorphisms in CNR1 have been associated with differences in obesity risk and possibly response to agents affecting the endocannabinoid system. While CB1 antagonist drugs (like rimonabant) are not on the market, the endocannabinoid tone can influence how patients respond to other appetite suppressants. Including CNR1 in the panel provides insight into the patient's appetite regulation pathway, potentially informing the aggressiveness of therapy needed for those with genetic predisposition to hyperphagia.
iii. TCF7L2 (Transcription Factor 7-Like 2)—e.g., variant rs7903146 (C>T). Rationale: TCF7L2 is a key gene in the Wnt signaling pathway and has the strongest common genetic association with type 2 diabetes. Its risk allele (T) is known to impair insulin secretion and incretin effect. Research demonstrates that TCF7L2 variation contributes to incretin resistance. For instance, carriers of the T allele have altered GLP-1 secretion and differential responses to diabetes medications. Although Camilleri et al. (2018) did not find a significant impact of rs7903146 on weight loss with GLP-1 RA in a small sample, other data suggest TCF7L2 genotype might influence whether a patient responds better to insulin sensitization (metformin) versus insulin secretion stimulation. Thus, including TCF7L2 helps tailor the choice between drug classes—e.g., a patient with TCF7L2 risk allele might benefit more from GLP-1 RA (to overcome incretin defects) or require higher doses to achieve the same effect.
iv. SLC47A1 (MATE1 transporter)—e.g., variant rs2289669 (G>A). Rationale: SLC47A1 encodes a transporter responsible for excreting metformin from the liver. The rs2289669 A allele has been associated with improved glycemic reduction on metformin, presumably due to higher intracellular metformin concentration in the liver (leading to greater drug effect on gluconeogenesis). By genotyping SLC47A1, the test can predict metformin efficacy: patients with the A allele are often better responders (greater HbA1c drop), whereas G/G homozygotes might have a more modest response. This information can guide whether metformin should be a cornerstone of the patient's weight management plan (metformin often yields modest weight loss but significant metabolic benefits in responsive individuals).
v. ATM (Ataxia Telangiectasia Mutated, locus often labeled C11 orf65)—e.g., variant rsl1212617.

Rationale: ATM is a gene identified by a large pharmacogenomic study as linked to metformin response. The C allele of rs11212617 near ATM was found to increase the odds of treatment success on metformin (OR ˜1.35). Although ATM's exact role in metformin's mechanism is not fully elucidated, it may involve ATM's interaction with AMPK signaling. In this panel, ATM rs11212617 serves as another predictor of metformin efficacy. If a patient carries the favorable allele, metformin could be particularly effective for weight stabilization/improvement of insulin resistance; if not, alternative or adjunct therapies might be considered sooner.
vi. SLC22A1 (OCT1—Organic Cation Transporter 1)—e.g., variant rs628031. Rationale: OCT1 is crucial for hepatic uptake of metformin. Loss-of-function polymorphisms in OCT1 (found predominantly in European populations) can reduce metformin uptake into the liver, thus diminishing its glucose-lowering effect. By including an OCT1 marker (rs628031 is one of several that affect OCT1 activity), the panel assesses another facet of metformin pharmacokinetics. Patients with reduced-function OCT1 alleles may require higher doses of metformin or might benefit from drugs that do not rely on OCT1 transport.
vii. UGT1A9 (UDP-glucuronosyltransferase 1A9)—e.g., UGT1A93 variant (such as rs72551330, Met33Thr)
Rationale: UGT1A9 is a liver enzyme that metabolizes many drugs, including certain SGLT2 inhibitors. For example, canagliflozin is inactivated via glucuronidation by UGT1A9 (and UGT2B4). Carriers of the UGT1A93 allele (which encodes a less active enzyme) have higher plasma drug levels for a given dose. In fact, individuals with a UGT1A93 allele exhibited a ˜45-54% increase in dose-normalized exposure (AUC) of canagliflozin in pharmacokinetic trials. While this might enhance efficacy somewhat, it could also raise the risk of side effects. Thus, knowing a patient's UGT1A9 status can guide SGLT2 inhibitor use—e.g., a patient with UGTIA93 might achieve therapeutic effects at a lower dose or should be monitored more closely for adverse effects like hypotension or UTI.
viii. DPP4 (Dipeptidyl Peptidase-4)—e.g., variants rs2909451 and rs759717. Rationale: DPP-4 is the enzyme target of DPP4 inhibitor drugs (sitagliptin, etc.). Genetic polymorphisms in DPP4 can lead to variations in baseline DPP-4 activity and how much it is suppressed by medication. For instance, one study found that individuals with the TT genotype of rs2909451 had higher DPP-4 enzyme activity even during sitagliptin treatment. While DPP4 inhibitor drugs generally cause weight-neutral effects, their glycemic efficacy might be affected by such variants. Therefore, DPP4 gene testing could identify patients who are likely good responders to DPP4 inhibitors versus those who might not get as much benefit, tilting the decision toward or away from that drug class. In particular, genotypes associated with high baseline DPP-4 activity indicate that a DPP-4 inhibitor may yield diminished glucose-lowering effect, guiding the clinician to consider a GLP-1RA or other drug instead for weight management.
ix. GNB3 (G Protein Beta-S Subunit)—e.g., variant rs5443 (C825T).
Rationale: The GNB3 825T allele has been historically associated with increased G-protein signaling efficiency and was linked to responses to certain medications (e.g., some studies in hypertension and obesity medication responses). In obesity, GNB3 825T has been associated with predisposition to obesity and possibly differential response to stimulants. Including GNB3 is exploratory, to capture an additional layer of how signaling differences might influence weight loss outcomes (for example, some evidence suggested that carriers of 825T respond differently to sibutramine, an older weight-loss drug).
x. OPRM1 (μ-Opioid Receptor)—e.g., variant rs1799971 (A118G, Asn40Asp).
Rationale: OPRM11 is included due to its relevance for therapies that involve the opioid pathway. The combination of naltrexone (an opioid antagonist) and bupropion relies in part on blocking opioid receptors to reduce cravings. The A118G polymorphism in OPRM1 is well known to alter receptor function—the Asp40 (G) allele reduces β-endorphin binding affinity and has been associated with a diminished response to naltrexone in alcohol dependence. Translating that to obesity, one can hypothesize that patients with the G allele may experience less appetite suppression or reward reduction from naltrexone, making the therapy less effective, whereas those with the A allele (normal receptor function) might benefit more. Hence, testing OPRM1 can inform whether naltrexone/bupropion (Contrave) is a genetically suitable choice for a given patient.
xi. CYP2B6 (Cytochrome P450 2B6)—e.g., variant CYP2B66 (often tagged by rs3745274 or rs2279343, but here represented by rs28399499 as a panel SNP). Rationale: CYP2B6 metabolizes bupropion to its active hydroxybupropion. Individuals with the CYP2B66 allele (which includes two common SNPs in linkage) have reduced enzyme activity and thus higher bupropion active metabolite levels. Clinical pharmacogenetic data show that CYP2B66 carriers had significantly better outcomes with bupropion in a smoking cessation trial (abstinence rates more than double that of placebo in *6 carriers, while non-carriers saw no benefit). This implies that the same genotype might modulate bupropion's effectiveness for weight loss (since bupropion's mechanism in weight loss is via appetite/craving suppression through dopamine/norepinephrine). Therefore, by testing CYP2B6, the method can indicate if a patient is likely to respond to bupropion (and at what intensity of effect, potentially guiding dose—e.g., CYP2B66 homozygotes might not need as high a dose to achieve a given plasma level).
xii. DRD2/ANKK1 (Dopamine D2 Receptor and associated Ankyrin repeat kinase)—e.g., rs1800497 (Taq1A) and rs6277 (C957T).
Rationale: The DRD2 gene's Taq1A polymorphism, actually located in the adjacent ANKK1 gene, is famous for its association with reduced D2 receptor density in the brain. The A1 allele (originally the “minor” allele) has been linked to higher risk of addictive behaviors, including overeating, due to blunted reward signaling. Another DRD2 SNP, C957T (rs6277), affects dopamine receptor availability as well. In obesity, these variants can identify individuals who have hedonic eating tendencies—those who may eat for reward more than hunger. From a treatment perspective, such individuals might particularly benefit from medications that modulate the dopamine/opioid reward pathways (again highlighting naltrexone/bupropion) or require additional behavioral support. Including DRD2 markers in the panel provides a genetic insight into the patient's craving profile, which, when combined with drug response markers, leads to a truly personalized therapeutic strategy. For example, a patient with high-risk DRD2 alleles might be steered toward Contrave as a first-line therapy to specifically target cravings, a decision supported by the genetic evidence that DRD2-related mechanisms underlie their obesity.
xiii. GRIK1 (Glutamate lonotropic Receptor Kainate 1)—e.g., variant rs2832407.
Rationale: GRIK1 encodes a subunit of the kainate receptor, which has been implicated in addictions and possibly appetite control. A particular intronic SNP, rs2832407, has been shown to moderate topiramate's effects on reducing heavy drinking; individuals homozygous for the C allele had significantly better outcomes in topiramate trials for alcohol use disorder. Topiramate, as part of phentermine/topiramate (Qsymia), aids weight loss partly by reducing appetite and cravings. Thus, a GRIK1 genotype might predict the degree of weight loss a patient would achieve on topiramate-containing therapy. If a patient has the responsive genotype, Qsymia could be very effective; if not, another medication might be preferred. By including GRIK1, the panel extends its predictive power to this FDA-approved combination therapy as well.
xiv. MC4R (Melanocortin 4 Receptor)—various mutations/polymorphisms.
Rationale: Though not tied to a specific medication response, MC4R is crucial for understanding a patient's baseline obesity risk. Mutations in MC4R are the most common cause of monogenic obesity, found in up to 5% of cases of early-onset severe obesity. Even common variants near MC4R have been associated with BMI in the general population. In this panel, MC4R serves as an obesity trait marker: if a patient carries loss-of-function MC4R mutations or risk alleles, it indicates a strong genetic drive to obesity (through increased appetite and reduced satiety). Clinically, such knowledge might prompt early, aggressive intervention or the consideration of novel therapies (e.g., setmelanotide, which is specifically for MC4R pathway deficiencies). At minimum, it helps contextualize the patient's weight struggle and set expectations—these patients may require combination therapy and lifestyle changes together to overcome their genetic propensity.
xv. FTO (Fat Mass and Obesity-Associated gene)—e.g., variant rs9939609 (T>A) among others.
Rationale: FTO was the first gene discovered by genome-wide association studies to have a major effect on common obesity. Carriers of risk alleles in FTO have higher average BMI and increased adiposity, reinforcing the need for sustained intervention; this has been replicated in many large studies across ethnicities. The effect size per allele is modest (a few kilograms), but it is a consistent predisposition factor. FTO's influence appears to be via appetite (increased food intake). In the panel, FTO serves as another trait indicator. A patient with homozygous FTO risk alleles might have a more robust body weight set-point, meaning they could be more resistant to weight loss—knowing this can encourage the clinician to closely monitor progress and possibly combine pharmacotherapy with more intensive lifestyle or adjunct interventions. Some evidence also suggests that high physical activity can blunt the effect of FTC risk alleles, so identifying these patients can motivate personalized lifestyle recommendations alongside drug therapy.

Additional variants discovered to be associated with anti-obesity drug response can be incorporated into the method without deviating from the invention's scope, reflecting the method's adaptability to emerging pharmacogenomic knowledge.

The proprietary panel consists of 16+ genes with multiple biomarkers, each included based on rigorous scientific evidence linking them to drug response or obesity traits. By evaluating all these markers together, the method delivers a multi-dimensional profile of the patient. This dual focus—pharmacogenetic markers (13 genes influencing medication response) plus obesity trait markers (3 genes related to BMI/appetite)—is a pioneering approach in obesity treatment, thereby enabling a precision-medicine approach to obesity treatment that is novel, non-obvious, and provides marked improvement in the personalization of therapy for weight loss. It recognizes that successful weight management depends on matching the right tool (medication) to the right patient, and that the patient's underlying biology (propensity for obesity, reward-driven eating, metabolic rate, etc.) will inform how aggressive or supportive the treatment needs to be.

Clinical Validation: The panel's design is rooted in associations that have been clinically validated in peer-reviewed studies. For instance, the importance of GLP1 R rs6923761 is supported by multiple independent investigations (including a 90-patient trial by de Luis et al., and a study in obese PCOS patients) which consistently indicate that genotype correlates with degree of weight loss on GLP-1 RAs. Similarly, the predictive value of SLC47A1 and ATM variants for metformin response has been confirmed through meta-analysis and large cohort studies. While not every variant in the panel is currently used in standard care, each has strong scientific plausibility and supporting data as a biomarker By doing so, it offers a novel and non-obvious combination: individually, some markers like TCF7L2 or FTO are known in the context of diabetes or obesity risk, but their joint use with drug-specific markers (GLP1 R, DPP4, etc.) to direct obesity pharmacotherapy has not been previously taught or suggested in the prior art.

In summary, the method of the present invention delivers several concrete advantages:
i. Improved Efficacy: Patients receive the drug most likely to work for them from the start, leveraging their genetic advantages (e.g., a GLPIR responder gets a GLP-1 RA, which might yield substantial weight loss in that genotype).
ii. Reduced Adverse Effects: By identifying slow metabolizers or those prone to side effects (like UGTIA9 variants leading to higher drug exposure), the method allows dose adjustments or alternative choices proactively.
iii. Time and Cost Savings: Both patients and healthcare systems benefit when effective therapy is identified sooner. Fewer clinic visits and medication changes are needed when the first choice is a success.
iv. Personalized Combination Therapy: The comprehensive nature of the panel means it can also highlight cases where combination therapy might be needed. For example, an individual with extreme genetic predisposition to obesity (MC4R, FTO) might be slated for combination treatment (drug+ intensive lifestyle or two drugs) right away rather than stepwise, conservative approaches.
v. Patient Engagement: Providing a patient with a personalized genetic report can also increase their engagement and adherence. Patients often find it motivating to know there is a biological reason for their struggles and that their treatment is tailored—it can enhance the patient-physician partnership and trust in the chosen regimen.

The detailed embodiments described here are not limiting. Variations of the panel could include additional genes (e.g., leptin or leptin receptor), or the method could be adapted to pediatric obesity (with appropriate consent and knowledge that genetic predispositions often manifest early). The method could also be provided as a service by laboratories, or integrated into an automated clinical decision support system in an electronic health record, where the genotype results trigger an alert with recommended medications.

By integrating decades of obesity and pharmacogenetic research into a single diagnostic tool, this invention paves the way for precision pharmacotherapy in obesity—a field that is increasingly important with the advent of many new obesity drugs and the recognition that one size does not fit all in weight management.

In one embodiment of the invention, the method can be implemented as follows (in a clinical workflow context): Sample Collection: A patient identified as needing pharmacological obesity treatment (for example, an adult with BMI 30, or 27 with comorbidities, who is a candidate for medication under current guidelines) provides a DNA sample. The sample can be peripheral blood or a buccal swab/saliva sample. Standard procedures for genetic testing are used to ensure DNA quality.

Genotyping/Sequencing: The laboratory performs genotyping of the specified variants. This can be done via a custom SNP genotyping array, targeted next-generation sequencing, or PCR-based assays. The included SNPs (like rs6923761 in GLP1 R, rs7903146 in TCF7L2, etc.) are assayed to determine whether the patient carries 0, 1, or 2 copies of the effect allele for each. Quality controls and known reference samples are used to validate the accuracy of genotypes.

Data Analysis: The patient's genotype data are input into a specialized algorithm or interpreted by a clinician according to a predefined interpretation chart. Each genotype is associated with a predictive insight. For instance:
i. If GLP1R rs6923761=A allele present→Predict: increased likelihood of good response to GLP-1 RA therapy.
ii. If TCF7L2 rs7903146=T/T (risk homozygote)→Predict: possibly reduced incretin effect; consider emphasizing GLP-1 pathway drugs or higher doses.
iii. If SLC47A1 rs2289669=A carrier→Predict: favorable metformin response (metformin likely to effectively improve glycemia and assist weight modestly).
iv. If ATM rs11212617=C carrier—Predict: higher chance of metformin success.
v. If UGTIA93 allele present→Predict: slow metabolism of canagliflozin; a lower dose might achieve same effect.
vi. If DPP4 rs2909451=TT→Predict: possibly high DPP4 activity; DPP4 inhibitor may be less effective (or need combination with another agent).
vii. If OPRM1 118G allele present→Predict: reduced naltrexone efficacy; consider alternative or higher dose if Contrave is used.
viii. If CYP2B66 genotype present→Predict: bupropion will be more effective (higher drug levels), but watch for side effects; if absent, bupropion effect might be average.
ix. If DRD2 A1 allele present (Taq1A)→indicating a dopamine signaling deficit Predict: patient has hedonic eating tendency; therapies targeting the brain's reward pathways to manage cravings (bupropion/naltrexone) may be particularly beneficial.
x. If GRIK1 C/C at rs2832407→Predict: strong topiramate response: Qsymia could yield significant weight loss.
xi. If MC4R mutation or risk allele→Predict: high genetic obesity drive; may require combination therapy and intensive lifestyle changes (and caution that standard doses might have less effect).
xii. If FTO risk alleles→Predict: patient prone to weight gain: ensure comprehensive management (diet/exercise) in addition to any drug, as the patient might regain weight if therapy is stopped.

In one embodiment of the invention, based on the above analysis, the clinician receives a report with recommendations. These recommendations can be tiered, for example:
i. Recommended First-Line Medication: The medication (or medication class) with the highest predicted efficacy for this patient and a favorable risk profile given their genetics. For instance, if the patient has a favorable GLP1 R genotype and no contraindications, a GLP-1 RA might be top-ranked. If the patient also has, say, an OPRM1 variant suggesting Contrave may be less effective, that drug might be ranked lower despite being an option in general.
ii. Alternative Options: Other medications that can be used if the first-line is not tolerated or if additional weight loss is needed. The profile might indicate moderate expected response for these. For example, a patient might have moderate predictions for metformin and DPP4 inhibitor—these could be alternatives or adjuncts for glycemic control if needed, but perhaps not primary for weight loss.
iii. Medications to Use with Caution or lower priority: If the patient's genetics predict poor response or higher risk for a certain medication, the report will flag this. For example, “SGLT2 inhibitor canagliflozin—patient has UGT1A9*3 allele leading to higher drug levels; use with caution (consider dose reduction or monitor closely)”, or “Phentermine/topiramate—patient's GRIK1 genotype is associated with lower response, so expected weight loss may be less than average.” None of these are absolute contraindications (genetics is one factor among many), but they inform priority.

In one embodiment, the invention provides a method of optimizing the dosage of an anti-obesity medication for a subject further adjusting the initial dose or titration schedule of the selected medication based on the subject's genotype-predicted metabolism of the drug.

In one embodiment, the invention provides a method for improving weight loss outcomes in a population of patients, wherein the overall result is a statistically significant increase in average weight loss or treatment success rate in the genetically guided group compared to an otherwise similar group of patients treated without genetic guidance.

In one embodiment of the invention, the clinician prescribes the recommended therapy, possibly at a tailored dose. For example, for a patient predicted to respond well to liraglutide, the physician starts liraglutide and expects above-average weight loss, thus can be aggressive in titrating knowing the benefit-risk is favorable. Conversely, if the test predicted only a mild response to that drug, the physician might opt for a higher dose alternative (such as semaglutide at a higher dose for weight management) or add an adjunct sooner (like combining metformin if the patient also had good metformin genes). The patient is monitored as usual (weight trajectory, metabolic parameters), but the expectation is that the initial choice is more likely to succeed, reducing the need for switching medications.

In a preferred embodiment of the invention, data from patients using this panel is collected to further refine the algorithm (machine learning correlates genotype patterns with actual outcomes, continuously improving the predictive power). This creates a feedback loop enhancing the test's accuracy as more real-world evidence accumulates.

### Exemplary Embodiments

Embodiment 1: A method for treating obesity and overweight comprising predicting a subject's response to one or more anti-obesity medications, comprising the steps:
i. obtaining a biological sample from the subject;
ii. analyzing the sample to determine the subject's genotype for a panel of genetic variants comprising at least GLP1R rs6923761, TCF7L2 rs7903146, CNR1 rs1049353, DPP4 rs2909451, DPP4 rs759717, SLC47A1 rs2289669, ATM rs11212617, UGT1A9*3 (rs72551330 or equivalent), CYP2B6 loss-of-function variant (e.g., rs3745274), GRIK1 rs2832407, DRD2/ANKK1 rs1800497, OPRM1 rs1799971, OCT1 rs628031, MC4R variant, FTO variant, and GNB3 rs5443 (optional);
iii. for each medication or class of interest, applying a predictive algorithm or lookup that incorporates the detected genotypes to determine an expected level of response (e.g., high, moderate, low) or risk of adverse effect;
iv. outputting a report that recommends one or more medications for weight loss and/or glycemic control that are predicted to be effective for the subject, and optionally recommends against certain medications predicted to be less effective or higher risk;
v. administering the one or more medications to the subject.

Embodiment 2: The method of Embodiment 1, wherein the medications considered include at least: GLP-1 receptor agonists, SGLT2 inhibitors, DPP-4 inhibitors, metformin, naltrexone/bupropion, and phentermine/topiramate. The method can be extended to any pharmacotherapy for obesity or obesity-related metabolic complications.

Embodiment 3: The method of Embodiment 1 or 2, wherein analyzing the sample comprises performing a multiplex genotyping assay selected from: (a) polymerase chain reaction (PCR) with allele-specific probes for each SNP, (b) hybridization to an oligonucleotide array, (c) targeted DNA sequencing, or (d) another molecular technique (like CRISPR-based detection or mass spectrometry of SNP loci). The assay is designed to have>99% analytical accuracy for each genotype.

Embodiment 4: A companion diagnostic kit for performing the method of Embodiment 1, comprising primers and probes for detecting each of the listed genetic variants, a positive control sample or DNA oligonucleotide representing known allele(s) for quality control, and instructions for interpreting the results in the context of obesity pharmacotherapy. The kit might also include interpretative guide or software (or access to a web portal) where the user can input raw genotype data and receive the interpretation automatically.

Embodiment 5: The genetic panel of this invention as used in pharmaceutical development. For instance, a company developing a new weight-loss drug could use this panel to identify genetic responders/non-responders in clinical trials (enriching the study or analyzing subgroup efficacy). Thus, the invention also contemplates a method of conducting a clinical trial or stratifying patient populations using the panel as a selection or randomization tool.

Embodiment 6: Integration with electronic health records (EHR). The results of the panel can be stored in the patient's EHR, and the system can provide point-of-care alerts. For example, if a provider attempts to prescribe a DPP-4 inhibitor to a patient who, according to their stored genotypes, is a predicted poor responder (DPP4 TT genotype, etc.), the EHR could flag this and suggest reviewing the genetic report. This synergy of genetic data and clinical decision support is part of the envisioned use of the invention.

### Examples

The genetic panel can be implemented via various molecular diagnostics: quantitative PCR with allele-specific probes, multiplex SNP genotyping (eg., TaqMan assays for each variant), targeted next-generation sequencing, or array-based methods. The panel may be offered as a laboratory-developed test or as part of a kit. The method generally comprises:
1. Sample Collection: Obtaining a DNA sample (e.g., saliva or blood) from the patient.
2. Genotyping: Testing for each variant on the panel. This could involve PCR amplification of relevant gene regions and detection of SNPs by probe hybridization or sequencing.
3. Data Analysis: Determining the patient's genotype for all targeted loci.
4. Interpretation: Using a knowledge database or algorithm to interpret how the specific combination of genetic results translates to drug response predictions. The output can be a report highlighting recommended therapies, likely effective drugs, drugs to use with caution (or at adjusted dose), and those less likely to be beneficial.

Example 1: A patient with GLPIR rs6923761 A allele and TCF7L2 T allele would likely respond well to GLP-1 RAs for weight loss, whereas their TCF7L2 genotype might caution that DPP-4 inhibitors alone won't sufficiently lower HbA1c [symbol] 29†L13-L21 [symbol]
The panel suggesting a GLP-1RA for this patient's obesity with careful monitoring of glycemic control or adding metformin.

Example 2: A patient carrying the DRD2 A1+ genotype (rs1800497 A allele) and FTO risk allele might have a strong reward-driven eating behavior. The panel indicates they are a good candidate for naltrexone-bupropion, as A1+ individuals showed significantly greater weight loss on NB (5.9% vs 4.2% in A1—in 8 weeks). Conversely, an A1—patient might be directed to other therapies first. Example 3: A patient with SLC47A1 rs2289669 AA genotype and ATM rs11212617 C allele is likely to have an excellent glycemic response to metformin [symbol] 31†L19-L27 [symbol] —this supports metformin as a foundational therapy. If the same patient also has CNR1 A allele, they might particularly benefit from adding a GLP-1RA for weight loss.

Example 4: A patient with MC4R risk variants and an extreme appetite phenotype (“genetic hyperphagia”) might need maximal appetite suppression—perhaps higher-dose GLP-1RA or even combination with phentermine-topiramate—and the panel flags this predisposition so clinicians can be more aggressive early on.

Example Clinical Scenario Case 1: Consider a 45-year-old female patient with a BMI of 37, struggling with obesity and prediabetes. The physician is contemplating either starting a GLP-1 RA (like semaglutide) or the combination naltrexone/bupropion, as both are viable options for weight loss in her case. The genetic test is performed and reveals the following genotype profile: GLP1 R rs6923761 A/G (one A allele present), TCF7L2 rs7903146 TIT (homozygous risk allele), DRD2 Taq1A A1/A2 (one A1 allele), OPRM1 A118G A/G (one G allele), SLC47A1 A/A (favorable metformin genotype), UGT1A9*3 negative (wild-type), and FTO risk alleles present. The report might interpret this as: “Patient has a genetic profile suggestive of strong response to GLP-1 receptor agonists (GLPIR A allele) and possibly diminished response to opioid antagonism (OPRM1 G allele). Her DRD2 status indicates some propensity for reward-driven eating, but the presence of the TCF7L2 risk allele and favorable metformin transport genotype suggest that addressing herinsulin/glucose axis (with GLP-IRA and/or metformin) may be particularly beneficial. Recommended plan: initiate a GLP-IRA (e.g., semaglutide) as primary therapy. Metformin can be added for its synergistic effects on weight and glycemia, given favorable genes (ATM, SLC47A1 positive). Naltrexone/bupropion is ranked lower priority due to the patient's OPRM1 genotype which may reduce naltrexone efficacy, although bupropion could still aid due to CYP2B6 status (not given in this example,). if needed, consider phentermine/topiramate later; patient's GRIK1 was not high responder genotype, so moderate outcome expected.” Armed with this information, the physician starts semaglutide and metformin. Indeed, the patient loses ˜15% of her weight over 6 months and improves her prediabetes, confirming the utility of the genetically guided choice. The alternative path—had she gone with Contrave and not responded well due to her OPRM1 variant—would have lost precious time; the genetic test helped avoid that.

Example Clinical Scenario Case 2: A patient with obesity (±type 2 diabetes or prediabetes) is considering pharmacotherapy for weight management. Before initiating therapy, the physician orders the genetic panel test.
1. Pre-test: The physician notes any clinical factors (e.g., contraindications) which might eliminate some drug options (e.g., uncontrolled hypertension might rule out phentermine-topiramate regardless of genetics [symbol] 35†L23-L31).
2. Testing: The patient provides a DNA sample. The lab runs the Anti-Obesity PGx Panel and returns a report.
3. Report Content: The report might have sections:
a. Genotype Summary: e.g., “GLP1 R G/A at rs6923761 (A allele present); TCF7L2: CIT (T present); CNR1: A/A (A allele homozygous); DPP4: T/T at rs2909451; DRD2 Taq1A: A/G (A1+).” For clarity, it could also denote them as “risk” or “benefit” alleles.
b. Medication Predictions: a table or narrative that integrates the genotypes. For example:
i. GLP-1RA: “Likely Excellent response. Patient carries GLP11R A allele associated with enhanced weight loss [symbol] 40†L1041-L1049. TCF7L2 genotype does not contraindicate (possibly neutral or slightly positive for weight loss). CNR1 genotype favorable for metabolic improvements [symbol] 42†L1146-L1154. Overall, GLP-1RA is txt use numb z33 fsi predicted to be highly effective.”
ii. DPP-4 inhibitor: “Reduced efficacy. Patient has DPP4 TT and CC P_{GP}-3₇,A_{RT}genotypes linked to high DPP-4 activity [symbol] 11†L121-L130 [symbol] [symbol] 12†L139-L148 [symbol] , and TCF7L2 T allele which in one study meant less HbA1c reduction [symbol] 29†L19-L27 [symbol] . A DPP-4i might yield suboptimal glycemic improvement; consider alternative or use only in combination.”
iii. Metformin: “Good response. SLC47A1 AA and ATM C allele suggest strong glycemic response [symbol] 11†L79-L87 [symbol] [symbol] 31†L19-L27 [symbol] . OCT1 variant present (which may slightly reduce hepatic uptake, but given other favorable markers, metformin is still recommended).”
iv. Naltrexone-Bupropion: “Highly favorable. DRD2 A1+ genotype—greater weight loss observed [symbol] 49†L7-L15 [symbol] ; OPRM1 genotype not detrimental. This points to patient being a good candidate for NB, especially if craving or emotional eating is a major issue.”
v. Orlistat (though not a focus, if included): genetic factors less clear; we might skip as it's not in the scope of PGx (main effect is behavioral).
vi. Phentermine-Topiramate: “Possible candidate. GRIK1 genotype indicates [interpretation], DRD2 A1+ also could benefit from phentermine's DA effects. Monitor for topiramate side effects since [e.g., if GRIK1 A allele means higher levels, may be titrate slowly].”
c. Obesity Trait insights: e.g., “Genetic profile suggests patient has a strong neurobehavioral component to obesity (DRD2, FTO risk alleles), as well as some metabolic predisposition (insulin resistance markers like TCF7L2). Thus, a combination of an appetite-regulating agent and a metabolic agent might be ideal.”
d. Plan: “Consider initiating liraglutide or semaglutide: if adequate weight loss not seen by 3 months, augment with naltrexone-bupropion (since genetics favor both). Ensure lifestyle interventions continue in parallel. If GLP-1 RA not tolerated, an SGLT2 inhibitor could be second choice given metformin likely effective (if not already on it). Avoid relying solely on DPP-4 inhibitors in this patient.”
4. Treatment & Follow-up: Guided by the report, the physician and patient choose a medication. The genetic test does not replace clinical judgment but greatly informs it. As treatment progresses, outcomes are monitored and adjustments are made. The genetic info remains useful lifelong—if new weight drugs come out, it can be re-interpreted in that context,

Example Clinical Scenario Case 3: 45-year-old female, BMI 37, prediabetes, struggles mainly with overeating (especially sweets: strong cravings). Genetic panel results: GLP1 R G/A, TCF7L2 C/T, CNR1 G/A, DPP4 T/C and G/C (heterozygous at both), SLC47A1 A/A, ATM A/C, UGT1A9 *3 absent, OCT1 AIG, CYP2B6 *1/*6, GRIK1 C/A, DRD2 A1+, OPRM1 A/G, MC4R risk present, FTO A/A (homozygous risk). Interpretation:
i. She has multiple markers for good response to GLP-1 RA (GLP1 R A; CNR1 A)—likely to lose weight well and improve metabolic markers [symbol] 40†L1029-L1038 [symbol] [symbol] 12†L184-L192 [symbol]
ii. She also has a profile suggesting reward-related eating (DRD2 A1+, FTO risk)—likely beneficial to incorporate NB for cravings.
iii. Start liraglutide 0.6→3.0 mg. Indeed, she loses 5% in 3 months but plateaus with still frequent cravings. Then add NB; over next 6 months, she loses an additional 8%, total ˜13% weight loss, which is significant for her.
iv. She was on metformin from prediabetes stage, which likely worked well (as predicted by ATM/SLC47A1).
v. DPP-4i were avoided, consistent with her genotypes suggesting low efficacy.

Example Clinical Scenario Case 4: 50-year-old male, BMI 34, type 2 diabetic for 5 years, main issue is lack of fullness (“hungry gut”), eats large meals. Genetic results: GLP1 R G/G (no A), TCF7L2 C/C, CNR1 G/G, DPP4 C/C and G/G (wild-type), SLC47A1 G/A, ATM A/A, UGT1A9 *3 present, OCT1 G/G (normal), DRD2 A1-, OPRM1 A/A, MC4R no risk, FTO T/A (one risk allele). Interpretation:
i. GLP-1 RA might still work (no adverse variants except may be missing the beneficial ones, but TCF7L2 CC is neutral or slightly favoring response).
ii. SGLT2 inhibitor could be interesting: he has UGT1A9*3, so canagliflozin levels would be higher [symbol] 12†L165-L172 [symbol] →may be more weight loss via glycosuria, but also watch for side effects. [symbol]
ii. No strong neurobehavioral drivers (DRD2 A1-, OPRM1 normal), so NB might be less impactful.
iv. Plan: Start semaglutide (since hungry gut—GLP-1 RA good for fullness). Monitor glucose (should improve given he's not TCF7L2 TT).
v. Indeed, semaglutide yields 10% weight loss. His A1 C drops from 8.0 to 6.5.
vi. Considering further loss, add canagliflozin; thanks to UGTIA9*3, he could see a good glycemic drop and a bit more weight (SGLT2i typically ˜2-Y3% weight loss, may be he gets 4%).
vii. Avoid phentermine due to age and mild hypertension risk factors; he didn't need NB due to lack of cravings and genetic suggestion that NB benefit would be average.

These scenarios show how the panel informs therapy choices and sequence. By having this genetic panel and method, practitioners can move beyond population averages to truly individualize obesity treatment.

A person skilled in the art can modify the invention described herein. However, it should be noted that this description relates to preferred embodiments of the invention and is provided for illustrative purposes only and should not be understood as limiting the invention. All obvious modifications in the spirit of the invention should be considered within the scope of the attached claims.

1. A method for treating obesity or overweight in a subject, comprising: (a) obtaining a biological sample from the subject, (b) analyzing the biological sample from the subject to determine the presence or genotype of one or more genetic variants in a predefined panel of genes, wherein the panel comprises variants in genes involved in anti-obesity drug response, including at least GLP1R, CNR1, TCF7L2, and DPP4, (c) selecting an anti-obesity medication or adjusting the dosage of said medication for the subject based on the detected genotype, such that the selected medication is predicted to have improved efficacy or safety for the subject's weight loss, and (d) administering the selected medication to the subject, wherein the presence of a specific genotype in the panel informs the choice of medication class best suited for the subject.

2. The method of claim 1, wherein the genetic panel further comprises variants in genes affecting metabolism of anti-diabetic medications used for weight management, including SLC47A1 (MATE1 transporter) and ATM (C11orf65), such that genotypes of said variants predict the subject's glycemic and weight response to metformin therapy.

3. The method of claim 1, wherein the step of selecting an anti-obesity medication comprises identifying a subject as a likely responder to a GLP-1 receptor agonist if the subject harbors a minor allele of a GLP1 R gene variant that is associated with enhanced weight loss response, or identifying the subject as a likely non-responder if said allele is absent.

4. The method of claim 3, wherein the GLP1R gene variant is rs6923761 and the presence of an A allele (encoding Ser\hat{ }168) indicates an increased likelihood of therapeutic efficacy with GLP-1 receptor agonists, prompting selection of a GLP-1RA as the preferred medication for the subject.

5. The method of claim 1, wherein if the subject's genotype includes a risk allele in TCF7L2 (rs7903146 T allele), the method further comprises selecting a therapy that enhances incretin signaling or insulin secretion, such as a GLP-1 RA or sulfonylurea.

6. The method of claim 1, wherein the panel further comprises variants in SLC22A1 (OCT1), SLC47A1 (MATE1), and ATM genes, and a subject's genotype in these genes is used to determine whether metformin will be effective.

7. The method of claim 1, wherein the panel further comprises the UGTIA93 allele (rs72551330 or an equivalent variant), and if the subject is identified as a carrier of UGTIA93, the method comprises either selecting a lower dose of an SGLT2 inhibitor or an alternative medication.

8. The method of claim 1, wherein the panel includes polymorphisms in the DPP4 gene (rs2909451 or rs759717) such that the subject's DPP4 genotype is used to predict responsiveness to DPP-4 inhibitor medications.

9. The method of claim 1, wherein the panel further comprises variants in OPRM1 and CYP2B6 genes, and the method is used to guide therapy with naltrexone-bupropion combination.

10. The method of claim 1, wherein the panel further comprises a variant in GRIK1 (rs2832407), and if the subject possesses a genotype indicating strong response, the method includes selecting a phentermine-topiramate therapy.

11. The method of claim 1, further comprising analyzing the subject's genotype for obesity-related trait genes including MC4R, FTO, and DRD2/ANKK1.

12. The method of claim 1, wherein the result of the genetic analysis is a stratification of the subject into a responder category for a particular drug or drugs, and the selected anti-obesity medication is chosen from the group consisting of: a GLP-1 receptor agonist, a biguanide (metformin), a DPP-4 inhibitor, an SGLT2 inhibitor, an opioid antagonist+ antidepressant combination (naltrexone+bupropion), sympathomimetic+anticonvulsant combination (phentermine+topiramate), or other pharmacological agents for weight loss.

13. The method of claim 1, wherein the subject is a human patient diagnosed with obesity or overweight, and the anti-obesity medication is an FDA-approved drug or combination for chronic weight management selected by the patient's genetic profile determined by said method.

14. The method of claim 1, wherein the analyzing of the biological sample comprises sequencing all or a portion of the subject's genome to identify said genetic variants.

15. The method of claim 1, wherein said method is implemented via software or algorithm that receives the subject's genotype data as input and automatically generates a report highlighting recommended therapies, likely effective medications, medications to use with caution or at adjusted dose, and those less likely to be beneficial.

16. A companion diagnostic kit for implementing the method of claim 1, comprising:
(i) a set of oligonucleotide primers or probes designed to detect the presence of the specific genetic variants in the panel including at least GLP1 R, CNR1, TCF7L2, and DPP4; (ii) reagents for performing DNA amplification or genotyping;
and (iii) an interpretative guide or software that correlates particular genotype combinations with recommended anti-obesity medications.

17. The companion diagnostic kit of claim 16, wherein the interpretative guide or software contains an algorithm that incorporates data from patients to output a report ranking potential medications.

18. A method of optimizing the dosage of an anti-obesity medication for a subject, comprising: (a) obtaining a biological sample from the subject, (b) analyzing the biological sample from the subject to determine the presence or genotype of one or more genetic variants in a predefined panel of genes, wherein the panel comprises variants in genes involved in anti-obesity drug response, including at least GLPIR, CNR1, TCF7L2, and DPP4, (c) selecting an anti-obesity medication or adjusting the dosage of said medication for the subject based on the detected genotype, such that the selected medication is predicted to have improved efficacy or safety for the subject's weight loss, (d) administering the selected medication to the subject and (e) further adjusting the initial dose or titration schedule of the selected medication based on the subject's genotype-predicted metabolism of the drug.

19. A method for improving weight loss outcomes in a population of patients, comprising: (a) obtaining a biological sample from the subject, (b) analyzing the biological sample from the subject to determine the presence or genotype of one or more genetic variants in a predefined panel of genes, wherein the panel comprises variants in genes involved in anti-obesity drug response, including at least GLP1R, CNR1, TCF7L2, and DPP4, (c) selecting an anti-obesity medication or adjusting the dosage of said medication for the subject based on the detected genotype, such that the selected medication is predicted to have improved efficacy or safety for the subject's weight loss, and (d) administering the selected medication to the subject, wherein the overall result is a statistically significant increase in average weight loss or treatment success rate in the genetically guided group compared to an otherwise similar group of patients treated without genetic guidance.
